(19) United States
(12) Patent Application Publication
(10) Pub. No.: US 2024/0238513 A1
(43) Pub. Date: Jul. 18, 2024

(54) INFUSION FILTER, AND INFUSION SET WITH INFUSION FILTER

(71) Applicant: B. Braun Melsungen AG, Melsungen (DE)

(72) Inventors: Michael Zerbes, Wabern (DE); Claudia Freitag, Gudensberg (DE); Andreas Katerkamp, Melsungen (DE)

(21) Appl. No.: 18/559,047

(22) PCT Filed: Apr. 29, 2022
(86) PCT No.: PCT/EP2022/061519

§ 371 Date: Nov. 4, 2023

(30) Foreign Application Priority Data
May 6, 2021 (DE) 10 2021 111 888.3

(51) Int. Cl. A61M 5/165; A61M 5/38

(52) U.S. Cl. CPC A61M 5/165; A61M 5/38

(57) ABSTRACT

An infusion filter and an infusion set that includes the infusion filter and an infusion line. The infusion filter has a housing defining a flow path between a housing inlet, couplable to a portion of the infusion line, and a housing outlet. A hydrophilic filter membrane is arranged in the flow path. The housing has, upstream of the filter membrane, an elbow for a first deflection of a flow along the flow path transversely to the main flow direction, and a baffle surface for a second deflection of the flow back into the main flow direction, ensuring that flow impinges on the filter membrane exclusively at the upstream membrane side. The infusion line has a first end portion with a connector for a container with an infusion solution, and a second end portion with a connector for a patient port or a further infusion line.

### CROSS-REFERENCE TO RELATED APPLICATIONS

This application is the United States national stage entry of International Application No. PCT/EP2022/061519, filed on Apr. 29, 2022, and claims priority to German Application No. 10 2021 111 888.3, filed on May 6, 2021. The contents of International Application No. PCT/EP2022/061519 and German Application No. 10 2021 111 888.3 are incorporated by reference herein in their entireties.

### FIELD

The disclosure relates to an infusion filter/air separator for an infusion line of an infusion set comprising a housing defining a flow path between a housing inlet and a housing outlet each coupleable to a portion of the infusion line, and a hydrophilic filter membrane arranged in the flow path and forming an air barrier, in particular after cessation of a flow/fluid flow along the flow path upstream of the infusion filter.

### BACKGROUND

In infusion treatments, a medical fluid or an infusion solution is supplied to a patient via an infusion line. One end/end portion of the infusion line is connected to a container for storing the infusion solution and the other end/end portion is connected to a patient port or an interface to another infusion line.

To exclude the possibility of harm to the patient, foreign bodies such as particles must be filtered in the infusion solution. It must also be ensured that no air enters the patient's body via the infusion line. If the container for storing the infusion solution runs empty, air from the container can enter the infusion line. Furthermore, air can also enter the infusion line through residual air from manifolds, drain cocks/shut-off valves or injection pieces, such as needleless or needle-based Y-site connections, especially if the injection pieces are not properly or completely vented.

Once foreign bodies and/or air have entered an infusion set, the infusion set must either be replaced with a new one or the air must be removed from the infusion line by appropriate handling of the infusion set to prevent harm to the patient, especially air embolisms.

Therefore, infusion filters are commonly used in conventional infusion systems. For example, EP 1 421 960 A1 shows an infusion filter which has a housing with an inlet and an outlet for connection to the infusion line. In order to be able to filter foreign bodies and prevent air from entering the infusion line, a hydrophilic filter membrane is arranged in the housing.

Furthermore, EP 2 500 051 A1 also shows a filter for use in an infusion set. Two filter elements, each of which has a hydrophilic filter membrane, are arranged inside a housing of the filter in order to be able to filter or separate foreign bodies and air from the infusion solution when an infusion solution flows through the filter.

However, such known infusion filters require a time-consuming priming process or respectively the time needed to prime the infusion set increases. In addition, with a small filter area, i.e. filters with small dimensions, only low flow rates can be realized and the infusion filters tend to clog/becoming occluded, so that conventional infusion filters usually have a bulky housing.

It is also known to use drip chambers and/or air separators for air separation in conventional infusion systems. For example, DE 299 21 086 U1 shows an infusion device with a drip chamber, from which an infusion line extends, and a hydrophilic filter membrane arranged in the drip chamber at the transition to the infusion line, which blocks the penetration of air into the infusion line in the moistened state.

Conventional drip chambers are located in the region of the container with the infusion solution, i.e. at the end portion of the infusion line away from the patient. This means that the risk of air entering through manifolds, valves or other connectors downstream of the drip chamber remains. In the case of multiple infusions, i.e. when the container with the infusion solution is changed, a large residual volume remains in the infusion line.

### SUMMARY

It is the object and aim of the disclosure to eliminate or at least reduce the disadvantages of the prior art.

In particular, the object of the disclosure is to optimize an infusion filter (air separator, infusion device) with hydrophilic filter membrane, in which the risk of air embolisms is largely eliminated and which is capable of delivering the flow rate/flow rate required for infusions with a relatively small size.

The object is solved with respect to a generic infusion filter according to the disclosure. The disclosure is thus based on the realization of counteracting turbulence formation in the flow or preventing turbulence formation in the flow.

Accordingly, the infusion filter is configured/adapted according to the disclosure such that the housing is configured such that the flow along the entire flow path is turbulence-free (laminar). In particular, the housing has, upstream of the filter membrane, a first flow-guiding element in the form of an elbow for a first deflection of a flow along the flow path transversely, in particular perpendicularly, to the main flow direction and a second flow-guiding element directly following thereafter in the form of a baffle surface oriented obliquely to the main flow direction for a second deflection of the flow back into the main flow direction, so as to ensure a flow against the filter membrane exclusively at its upstream membrane side. I.e. the housing upstream of the filter membrane has a first flow-guiding element in the form of an elbow for a first deflection of a flow along the flow path transversely, in particular perpendicularly, to a main flow direction and a second flow-guiding element in the form of an inclined baffle surface (inclination/ramp/bevel) for a second deflection of the flow in the main flow direction. In this way, the flow within the housing can be diverted gently, i.e. without provoking detachments or causing dead spaces/dead water areas/detachment bubbles, and optimal flow to the filter membrane can be ensured.

Advantageous embodiments are explained below.

In an advantageous embodiment, the, preferably plate-shaped, filter membrane may be arranged in the housing such that it extends substantially in or along the main flow direction. That is, the filter membrane may be arranged in the housing such that a normal to one of the front surfaces of the filter membrane is perpendicular to the main flow direction. In other words, the filter membrane may be arranged in the housing such that the flow through the filter membrane is substantially perpendicular to the main flow direction.

According to a further development in accordance with the disclosure, the housing may have a third flow-guiding element downstream of the filter membrane in the form of an elbow, which deflects the flow flowing substantially vertically through the filter membrane into the main flow direction. This also prevents the formation of dead water areas downstream of the filter membrane, in which residual air bubbles can collect.

In other words, the flow can be guided along the flow path via flow-guiding elements, preferably in the form of inclinations and radial/rounded deflections. In other words, flow-guiding elements for guiding along the flow path can be configured as inclination/inclined baffle surface/ramp/bevel or radii/rounded deflections, i.e., the flow guidance within the infusion filter may be formed without corners or undercuts, so that no dead water areas can form, at which air bubbles can settle or form. In particular, the deflection regions in the region of the housing inlet and the housing outlet can be configured in the form of an elbow (a quartered sphere or quarter torus), which allows a particularly gentle deflection of the flow, even by more than 45°, especially 90°. A radius of the elbow can preferably be between 1 mm and 2 mm, in particular 1.5 mm. Furthermore, the inclined baffle surface can be formed at an inflow region, which opens to a cavity formed in the housing for receiving the filter membrane, which, similar to the deflection regions, enables a gentle deflection and optimized inflow to the filter membrane. An angle of inclination can be adapted to the inflow area as desired. Preferably, the angle can be between 5° and 35°, in particular between 100 and 20°, and especially preferably around 15°.

According to an advantageous embodiment, the housing inlet and the housing outlet may have a receiving portion/receiving region for receiving the respective infusion line portion, and the receiving portions can be adapted to dimensions of the infusion line portions in such a way that, in the coupled state, inner diameters of the portions of the infusion line end flush with the surface of the receiving portions. Particularly preferably, the receiving portions may each have a circumferential step as a stop for the infusion line, the height of which substantially corresponds to a wall thickness/thickness of the infusion line, i.e. when the infusion line is pushed/inlayed/inserted into the infusion filter, no gap or steps form at the interface/transition between the infusion line and the infusion filter, which prevents the formation of a gap/step at this interface, which in turn can prevent residual air bubbles from accumulating.

Furthermore, according to the disclosure, it can be particularly advantageous if the housing has a lower housing part and a housing lid that can be form-fitted and/or force-fitted and/or firmly bonded to each other and thus form a cavity inside the housing.

In addition, it can be useful if the infusion filter according to the disclosure has an air-permeable vent filter arranged in the housing, which is connected to an outer housing side via an opening formed in the housing. In this way, residual air bubbles that accumulate/settle inside the housing, in particular after the flow upstream of the infusion filter has stopped, can flow out/flow away. At the same time, the vent filter prevents foreign bodies from entering.

In an advantageous further development, the housing may have a support structure for receiving the filter membrane. The support structure can have a closed, preferably circular, crosspiece following the geometry of the filter membrane and support elements, in particular ribs and knobs (support points), arranged within this crosspiece. The support structure ensures that the filter membrane is held securely and also prevents the formation of possible turbulence or residual air bubbles. It can be particularly useful if the ribs are arranged in the main flow direction aligned downstream of the filter membrane directly following the latter and the knobs are placed downstream of the ribs and preferably transversely offset to the ribs with respect to the main flow direction.

Furthermore, it is preferred if the filter membrane has a bubble pressure between 0.2 and 0.45 bar and/or an area below 2.0 cm², preferably below 1.7 cm², which allows a high flow rate with sufficient filter/barrier effect.

In addition, it can be useful if the filter membrane is configured in such a way that the hydraulic permeability for water (water flow rate) at a pressure of 0.1 bar is at least 120 ml/min, preferably at least 140 ml/min. Such a filter membrane allows the infusion filter according to the disclosure to be used in standardized infusion lines and for all infusion treatments, since the water flow rate exceeds the normatively required value of 1000 ml/10 min (ISO 8536-4).

According to a preferred embodiment, the filter membrane may be composed of a plurality of parallel tubes. Alternatively, the filter membrane may also be constructed from a block with gaps, from lamellae or an open porous material, in particular a membrane, a felt or foam. In such a filter membrane, the capillary forces created/prevailing ensure the function/action of the filter membrane as an air barrier due to a capillary flow stop.

Furthermore, the disclosure relates to an infusion set with an infusion line, the first end portion of which has a connector for a container with an infusion solution and the second end portion of which has a connector for a patient port or a further infusion line, and an infusion filter arranged/interposed in the infusion line according to the disclosure. The infusion filter may be arranged in the direction of flow of the infusion solution from the first end portion to the second end portion directly upstream of the second end portion or may be integrated directly in the connector for the patient port, i.e. the residual volume remaining in the infusion line is small and a possible formation/accumulation of residual air bubbles in further components possibly arranged downstream (between infusion filter and connector for patient port), such as shut-off valves or the like, can be avoided. Furthermore, in an infusion set according to the disclosure, it can be useful if a drip chamber arranged in the infusion line and/or a clamping device arranged on the infusion line is provided.

In other words, the disclosure relates to a low-cost infusion filter for an infusion set that allows an air stop function, i.e., an air barrier, to be routed from a drip chamber to the end of the infusion line to reduce residual volume and eliminate air from other sources, such as Y-connector pieces, shut-off valves, or venting. In this regard, the disclosure is characterized in that the infusion filter eliminates/separates air from an infusion line and automatically stops the flow. Here, the air separation takes place near the end of the infusion line in close proximity to the patient, resulting in decreased/low residual volume and efficient air separation. In addition, the infusion filter is quick and easy to prime, which eliminates the need for a special priming procedure or additional steps in handling by a user. Furthermore, the infusion filter also sees a high flow rate with minimized size and very low residual volume, as well as effective retention of particles at least 3 μm in size (particle size ≥3 μm). A trade-off between the specifications of the hydrophilic fluid filter membrane and the specified area of the hydrophilic fluid filter membrane provides a high flow rate combined with a very small and non-barrier size of the infusion filter. For this, the hydrophilic fluid filter membrane has a bubble pressure between 200 and 450 mbar and the area of the hydrophilic fluid filter membrane is below 2.0 cm², in particular below 1.7 cm². Infusion sets with an integrated infusion filter thus have a water flow rate of at least 120 ml/min, preferably 140 ml/min, at a pressure difference of 0.1 bar. The housing of the infusion filter has a design that allows rapid priming in any position of the infusion filter, without a special priming procedure or additional steps in handling by the user. The priming time of the infusion filter is less than 15 s, preferably less than 10 s, more preferably less than 5 s. The residual volume of the infusion filter is below 0.2 ml, preferably below 0.16 ml. Here, the infusion filter is primarily configured as a set of hydrophilic capillaries, and not as an air filter for germ retention. The set of hydrophilic capillaries is intended to retain air and particles with a particle size of at least 3 μm. Thus, the disclosure allows rapid priming of the infusion line and of the infusion filter in any orientation without a special priming procedure, saving time for the user during preparations. The small dimensions of the infusion filter housing, in turn, result in reduced force on the infusion line, help prevent sticking or snagging of the infusion filter in or on other infusion lines or other objects, and result in low residual volume, resulting in minimal loss of medication at the end of the infusion. The high water flow rate ensures rapid volume exchange in emergency situations. Due to the efficient particle retention, the particle load on the patient during infusion treatment can also be reduced. In addition, the efficient air separation in (close) proximity to the patient helps to prevent air ingress into the patient during infusion treatment. For this purpose, the housing of the infusion filter has an optimized flow path without dead spaces to achieve fast and air bubble-free priming. Due to this design, bubble-free priming works independently of the orientation of the infusion filter and does not require any special priming procedure or additional handling by the user. In one embodiment, the infusion filter may be the hydrophilic fluid filter membrane, which has a bubble pressure between 200 and 450 mbar, an air flow rate greater than 5.5 LSL (ft3/ft2/min), preferably 6.5 LSL, at a pressure of 125 Pa in the non-wetted (dry) state, a water flow rate of more than 1200, in particular 1400, ml/cm²/min at a pressure of 10 psi, and an area of less than 2.0 cm², in particular less than 1.7 cm².

In other words, the installation size and internal cavities of the infusion filter are kept as small as possible. There are no sharp-edged 90° transitions. All transitions can be configured as radii or inclinations. In addition, an inclination in the housing lid can help to direct the fluid flow accordingly and without turbulences to the fluid filter membrane. In the housing, the region between the inlet opening and the edge of the cavity may be completely filled with plastic to prevent the formation of a dead space. Furthermore, supports for the fluid filter can prevent possible turbulences and residual air bubbles when priming the filter. In the infusion filter according to the disclosure, a combination of ribs and individual support points/knobs can be selected for these supports. Conventional infusion filters usually have only ribs, between which residual air can still adhere. The dimensions and sizes of the housing inlet and of the housing outlet can be adapted to the dimensions of the infusion line (tube dimensions) so that there are no shoulders/steps or gaps between the infusion line/tube and the tube attachment, which prevents residual air bubbles from adhering. The deflection to the housing may also be spherical. The inclination in the housing lid can direct the fluid flow toward the fluid filter membrane without turbulences or ‘air pockets’. The angle of inclination can be adapted to the distance between the edge of the cavity and the inlet opening and can be between 10° and 20°, and in particular 15°. In the lower housing part, the corresponding region may not be configured as an inclination, but may be completely filled with plastic. This combination of inclination in the housing lid and the ‘filled’ region in the lower housing part can direct the fluid flow accordingly and thus ensure that the fluid filter membrane can be optimally hit by the flow and wetted during priming. The optimal flow during wetting can in turn prevent the entrapment of air bubbles downstream of the filter membrane during filling of the infusion filter.

In yet other words, the disclosure relates to an infusion filter with a high (water) flow rate of more than 120 ml/min, preferably more than 140 ml/min, at a pressure of 0.1 bar in combination with a small size of the infusion filter or a small area of the fluid filter membrane. The infusion filter according to the disclosure is furthermore also suitable for infusion solutions with a higher viscosity, such as glucose solution or lipid emulsion. To achieve bubble-free filling/priming of the infusion filter, no special priming procedure or additional handling steps by the user are required. The bubble-free filling of the infusion filter works independently of its position/orientation in space. The infusion filter according to the disclosure has an optimized flow path, whereby there are no dead water areas inside the assembled infusion filter where air bubbles get stuck during the filling of the infusion filter. In addition, the position of the inlet and outlet openings in the cavity are chosen to ensure optimal flow of the infusion solution to the filter membrane without entrapment of air bubbles during filling.

Further advantages are that the infusion set can be changed from an empty infusion container to a new, full infusion container without manipulation by the user. Furthermore, position-independent venting is made possible. When boluses are delivered through improperly vented bolus injection ports, valves or three-way stopcocks, air is eliminated and potential particles are retained during injection delivery. This offers both increased user comfort and increased patient safety.

### BRIEF DESCRIPTION OF THE DRAWINGS

The disclosure is explained in more detail below with reference to preferred configuration examples with the aid of Figures.

FIG. 1 shows a perspective view of an infusion filter according to a preferred configuration example;

FIG. 2 shows an exploded view of the infusion filter according to the preferred configuration example;

FIG. 3 shows a perspective view of a lower housing part of the infusion filter according to the preferred configuration example;

FIG. 4 shows a longitudinal sectional view of the lower housing part of the infusion filter according to the preferred configuration example;

FIG. 5 shows a perspective view of a housing lid of the infusion filter according to the preferred configuration example;

FIG. 6 shows a bottom view of the housing lid of the infusion filter according to the preferred configuration example;

FIGS. 7a and 7b show schematic diagrams of the mode of action of a hydrophilic filter membrane of the infusion filter according to the preferred configuration example;

FIG. 8 shows a longitudinal sectional view of the infusion filter connected to an infusion line according to the preferred configuration example; and

FIG. 9 shows a perspective view of an infusion set with the infusion filter according to the preferred configuration example.

### DETAILED DESCRIPTION

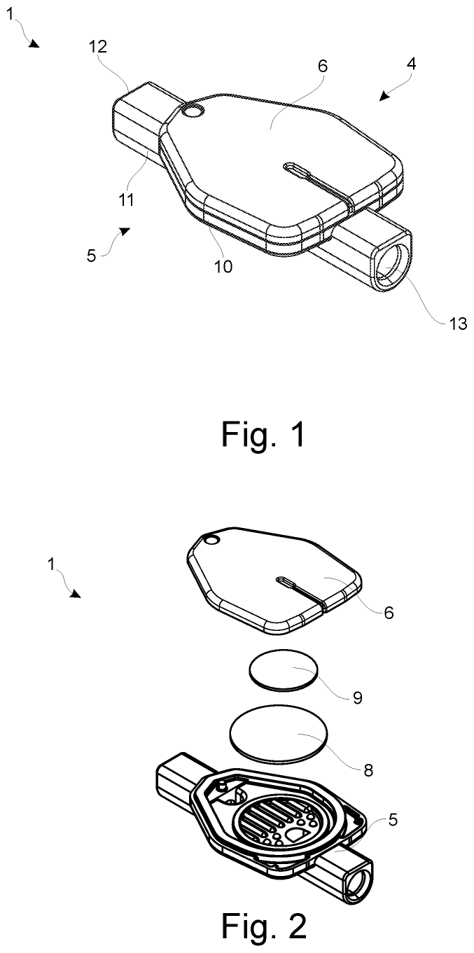

FIG. 1 shows a perspective view and FIG. 2 an exploded view of an infusion filter/air separator 1 according to a preferred configuration example for an infusion set 2 described in more detail below, i.e. the infusion filter 1 is arranged to be connected to an infusion line 3 carrying an infusion solution or to be interposed in the infusion line 3.

As shown in FIG. 1, the infusion filter 1 has a housing 4 with a lower housing part 5 and a housing lid 6 that is connected in a form-fitted and/or force-fitted manner to the lower housing part 5.

Inside the housing 4, i.e. in the region of a cavity 7 (hollow space) formed between the lower housing part 5 and the housing lid 6, a hydrophilic (fluid) filter membrane 8 and a vent filter 9 are arranged, as shown in FIG. 2. As described in more detail below, the filter membrane 8 acts as an air barrier and the vent filter 9 allows air that collects in the region of the cavity 7 to be transported to an outer side of the housing 4. The filter membrane 8 thus divides the housing 4 into an upstream housing portion 4a and a downstream housing portion 4b.

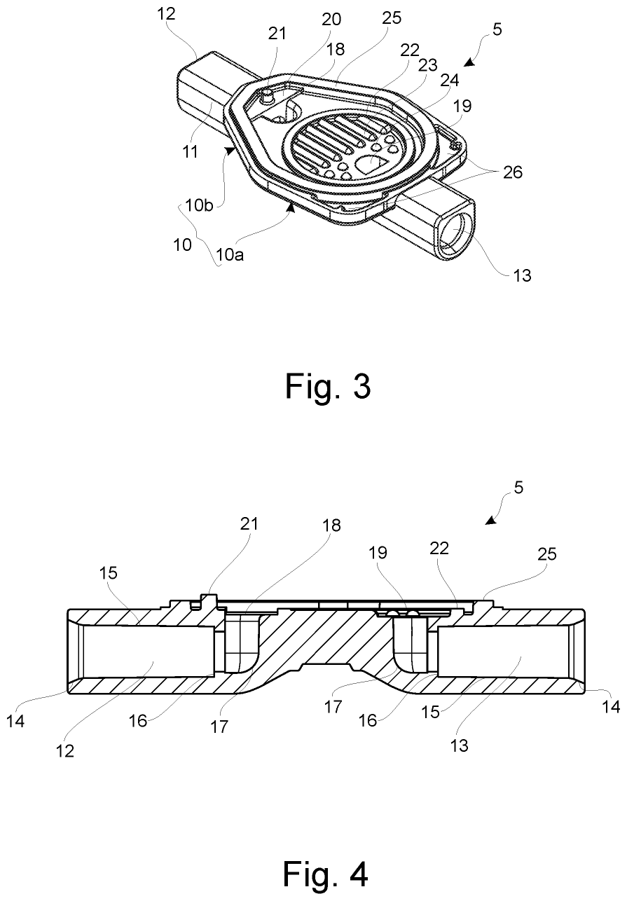

FIG. 3 shows a perspective view of the lower housing part 5 of the infusion filter 1 according to the preferred configuration example. The lower housing part 5 has a main body 10 which, in a top view, is configured in the form of an escutcheon, i.e., the main body 10 has a symmetrical polygonal shape with a wide first portion 10a and a tapering second portion 10b. Furthermore, the lower housing part 5 has a beam body 11 disposed on an underside of the main body 10 and extending in the longitudinal direction beyond the first and second portions 10a, 10b of the main body 10. In other words, the lower housing part 5 has the main body 10 and the beam body 11, the beam body 11 being longer than the main body 10 in its extension direction and narrower than the main body 10 transversely to its extension direction.

Furthermore, as shown in FIG. 4, the beam body 11 has an opening on each of its two front surfaces, each of which is in fluid communication with the cavity 7 formed between the lower housing part 5 and the housing lid 6. Accordingly, the beam body 11 forms a housing inlet 12 and a housing outlet 13, wherein the housing inlet 12 is formed on the side of the second portion 10b with respect to the main body 10 and the housing outlet 13 is formed on the side of the first portion 10a.

As mentioned above, according to the preferred configuration example, the infusion filter 1 is arranged to be fluidically connected to the infusion line 3. That is, the housing inlet 12 can be connected to an upstream portion of the infusion line 3, whereas the housing outlet 13 can be coupled to a downstream portion of the infusion line 3. For this purpose, the upstream portion and the downstream portion are pushed into the housing inlet 12 and the housing outlet 13, respectively. For easier handling during inlaying/insertion of the infusion line 3, the housing inlet 12 and the housing outlet 13 each have an insertion structure 14 configured as a chamfer/inclination. A respective receiving region 15 for the respective portions of the infusion line 3 adjoins this insertion structure 14 in the insertion direction. At the end region of the receiving regions 15 facing away from the insertion structure 14, a respective circumferential step 16 is formed as a stop for the portions of the infusion line 3. As shown in FIG. 7, a height of the step 16 corresponds substantially to a wall thickness of the infusion line 3, i.e. the receiving regions 15 are adapted to the infusion line 3 in such a way that no steps/shoulders are formed when the infusion line 3 is inserted, thus preventing possible turbulences that could lead to the formation of air bubbles by avoiding shoulders/steps along the flow path. In other words, dimensions of receiving regions 15 of housing inlet 12 and of the housing outlet 13 are adapted to dimensions of infusion line 3 such that there are no shoulders or gaps between infusion line 3 and receiving region 15. In yet other words, the receiving regions 15 of the housing inlet 12 and the housing outlet 13 are adapted to the dimensions of the infusion line 3 in such a way that in the inserted state, i.e. when the infusion line 3 has been inserted into the infusion filter 1, the inner diameters of the infusion line 3 are flush with the surface of the steps 16. This prevents residual air bubbles from settling here.

Adjacent to step 16, i.e. on the side of housing inlet 12 and housing outlet 13, is a deflection region/elbow 17, via which the flow of the infusion solution is deflected by approximately 90°. As shown in FIG. 4, the deflection regions 17 are rounded or have the shape of a quartered sphere, one open cut surface of which is oriented toward the housing inlet 12 and the housing outlet 13, respectively, and the other open cut surface of which is arranged to form a right angle to the one open cut surface. In yet other words, the deflection in the housing 4 is spherical, so that the flow can be deflected without creating turbulences or dead water areas.

As can be seen in FIG. 4, the infusion filter 1 according to the preferred configuration example is configured in such a way that the elements described above, i.e. the insertion structure 14, the receiving region 15, the step 16 and the deflection region 17, are the same for the housing inlet 12 and the housing outlet 13. That is, the housing inlet 12 is substantially symmetrical to the housing outlet 13 with respect to a center plane of the infusion filter 1.

On the side of the housing inlet 12, an inlet opening 18 opening towards the cavity 7 adjoins the deflection region 17, whereas on the side of the housing outlet 13, an outlet opening 19 opening towards the cavity 7 is formed. In a top view of the lower housing part 5, both the inlet opening 18 and the outlet opening 19 are in the form of a half ellipse, with the radii of the half ellipses being aligned with each other.

As can be seen in FIG. 3, a mounting portion 20 with a projecting mounting pin 21 is formed on a side of the inlet opening 18 facing away from the outlet opening 19. On a side of the inlet opening 18 facing the outlet opening 19, a circular crosspiece 22 is arranged, within which the outlet opening 19 as well as ribs 23 aligned in the extension direction of the beam body 11 and individual knobs 24 are formed. As described in more detail below, the crosspiece 22, ribs 23 and knobs 24 serve as a support structure for the filter membrane 8 received in the lower housing part 5. This support structure for the filter membrane 8 can prevent possible turbulences and residual air bubbles during priming of the infusion filter 1.

In a rim region of the main body 10, a further crosspiece 25 is formed which follows the shape of the main body 10 in the region of the second portion 10b and is semicircular in the region of the first portion 10a. In other words, in the region of the second portion 10b, the crosspiece 25 is offset inward from the edge of the main body 10 in such a way that a step of constant width results. In the region of the first portion 10a, the crosspiece 25 describes a semi-circular shape so as to expose shoulder regions 26 of the main body 10 which, as will be described in more detail below, serve for receiving the housing lid 6.

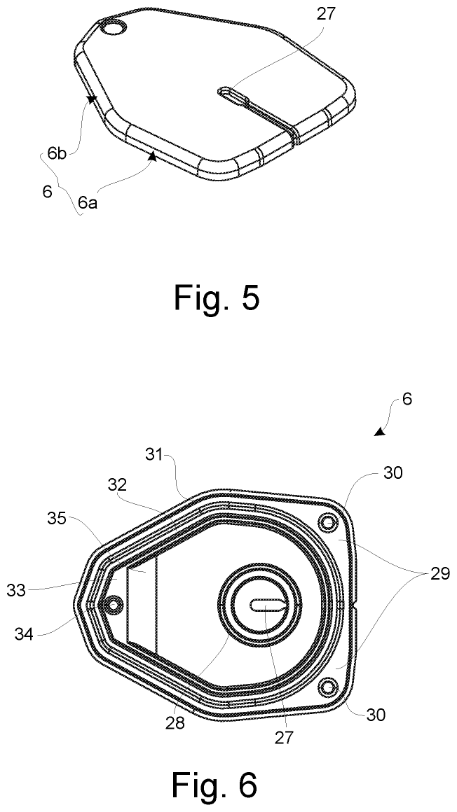

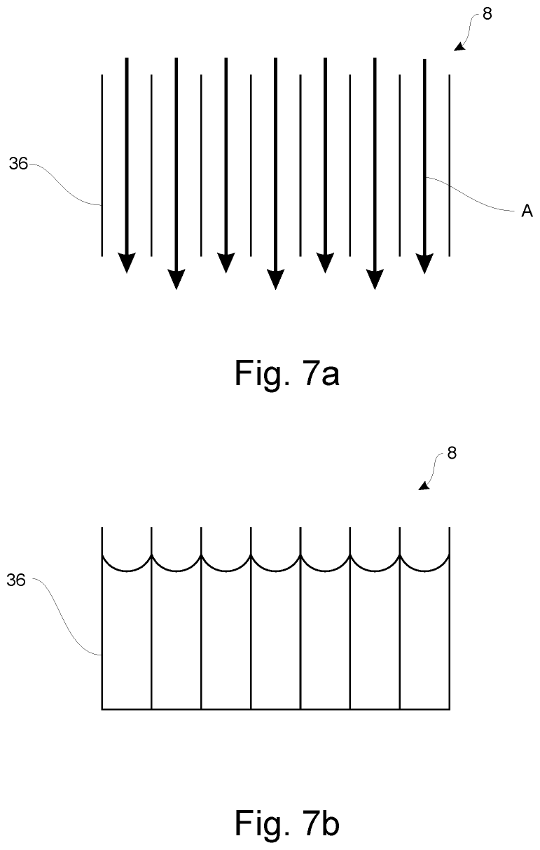

FIGS. 5 and 6 show views of the housing lid 6 of the infusion filter 1 according to the preferred configuration example. The housing lid 6 has, similar to the main body 10 of the lower housing part 5, an escutcheon shape with a first, wide portion 6a and a second, tapered portion 6b. In the region of the first portion 6a, a continuous opening in the form of an elongated hole (vent opening) 27 is formed in the center. As can be seen in FIG. 6, on an inner side of the housing lid 6, which faces the cavity 7 in the assembled state of the infusion filter 1, a circular crosspiece 28 is formed around the elongated hole 27 and serves for receiving the vent filter 9, as will be described in more detail below. In shoulder regions 29 of the first portion 6a of the housing lid 6, mounting pins 30 are arranged in each case, which cooperate with shoulder regions 26 of the main body 10 during assembly of the infusion filter 1 according to the preferred configuration example.

In a rim region of the housing lid 6, a first crosspiece 31 of triangular cross-section is formed, which substantially follows the shape of the crosspiece 25 of the main body 10 and rests on it or is pressed/pushed against it during assembly of the infusion filter 1, thus forming a seal. In other words, the crosspiece 25 of the main body 10 and the crosspiece 31 of the housing lid 6 seal the lower housing part 5 and the housing lid 6 against each other.

Within the first crosspiece 31, a circumferential second crosspiece 32 following the shape of the first crosspiece 31 is formed. In the region of the second portion 6b, i.e. at the tapered end portion of the housing lid 6, a mounting region 33 with a mounting opening 34 in the form of a blind hole is arranged so that the mounting pin 21 engages in the mounting opening 34 when the infusion filter 1 is mounted according to the preferred configuration example. Both in the lower housing part 5 and in the housing lid 6, the mounting region 20 or 33 between inlet opening 18 and crosspiece 25 is completely filled with plastic, i.e. formed from solid material. This prevents the formation of a dead water area.

On a side of the mounting region 33 facing the elongated hole 27, a ramp/chamfer/inclination 35 is formed, which, as will be described in more detail below, ensures a controlled, turbulence-free redirection of the flow through the infusion filter 1, i.e. the inclination 35 in the housing lid 6 helps to direct the flow accordingly and guide it towards the filter membrane 8 without turbulences. In the infusion filter 1 according to the preferred configuration example, the angle of inclination 35 is approximately 15°. However, the inclination 35 can of course also assume a different angle. In particular, the angle of inclination 35 is adapted to the distance between the crosspiece 25 and the inlet opening 18.

The housing lid 6 can be equipped with one or more vent grooves (venting grooves) on the outside. The vent grooves prevent the vent opening 27 from closing (e.g. by fixing the air separator 1 to the patient or by the patient's position). The vent grooves lead from the vent opening 27 to the outer edges of the housing lid 6. To prevent pressure points on the skin, the housing lid 6 can be configured without raised ribs or other raised regions.

FIGS. 7a and 7b schematically illustrate the operating principle of the filter membrane 8. In the preferred configuration example, the filter membrane 8 is constructed as a set of hydrophilic capillaries, which can be regarded as a plurality of parallel tubes 36 packed together. Now, when a liquid flows through the filter membrane 8, it flows through the parallel tubes 36 (arrows A in FIG. 7a). As mentioned above, as a liquid, the infusion solution flows in the infusion set 2 due to gravity. I.e. the weight of the liquid provides the pressure required for flow.

If no more liquid flows, for example because no more infusion solution is present and/or a patient has been administered a full dose of infusion solution, the flow through the capillaries comes to a standstill and a liquid level remains at an upper end of the capillaries, i.e. the filter membrane 8 remains substantially completely wetted or in contact with the infusion solution. Due to capillary forces, as shown in FIG. 7b, a concave meniscus or concave bulge is formed at the respective upper end portions of the individual capillaries at the liquid surface. That is, when the flow of liquid upstream of the filter membrane 8 stops, the liquid adheres tightly between the capillaries, forming a barrier to air. In other words, when the flow of the infusion solution upstream of the filter membrane 8 stops, the filter membrane 8 prevents air from entering the downstream portion of the infusion line 3. Depending on the diameter of the tubes 36 or resp. of the capillaries, the adhesive force between the liquid and the surface of the tubes 36, and the surface tension (surface energy) of the liquid, the capillary forces are large enough to counteract the weight force of the liquid downstream and to prevent the liquid from flowing into the downstream portion of infusion line 3 or out of the infusion filter 1. I.e. the filter membrane 8 represents an air barrier via capillary flow stop.

In the preferred configuration example, the filter membrane 8 has a bubble pressure between 0.2 and 0.45 bar and an area below 2.0 cm², in particular below 1.7 cm². The infusion set 2 with the infusion filter 1 according to the preferred configuration example thus has a flow rate above 120 ml/min, preferably 140 ml/min, at a pressure difference of 0.1 bar.

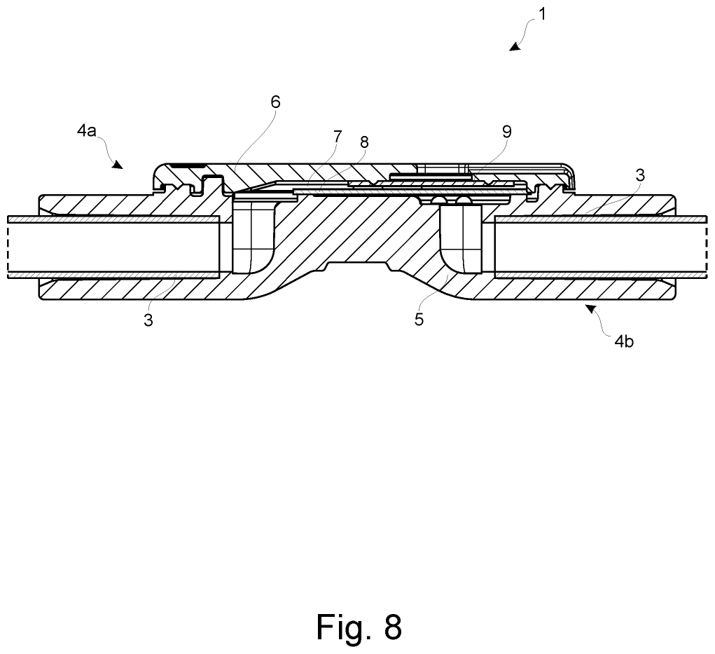

The plate-shaped vent filter 9 is made of a conventional, air-permeable filter material and, as can be seen in FIG. 8, is accommodated in the housing lid 6. One front surface of the vent filter 9 is oriented towards the cavity 7 and another front surface lies against the inside of the housing lid 6. In the housing lid 6, as mentioned above, the elongated hole 27 is formed, via which the inner housing side is connected to the outer housing side. As can be seen in FIG. 1, the vent filter 9 is accommodated in the housing lid 6 in such a way that the vent filter 9 completely covers the elongated hole 27. Thus, air which collects in the cavity 7, as described in more detail below, can flow through the vent filter 9 and the elongated hole 27 to the outer housing side. I.e. the vent filter 9 and the elongated hole 27 serve to vent the infusion filter 1.

In FIG. 8, the infusion filter 1 according to the preferred configuration example is shown in the assembled state with the infusion line 3 inserted. When the infusion solution flows into the infusion filter 1 via the infusion line 3 and the housing inlet 12, it first enters the infusion filter 1 at the interface, i.e. the step 16 which is flush with the inner diameter of the infusion line, and is deflected in the deflection region 17 towards the cavity 7, where it flows into the cavity 7 through the inlet opening 18. The inclination 35 on the housing lid 6 ensures turbulence-free deflection and thus optimum flow to/wetting of the filter membrane 8. As long as infusion solution is present, it flows over the filter membrane 8, i.e. through the tubes 36, and is guided to the outlet opening 19 with the aid of the ribs 23 and knobs 24. The infusion solution flows through the outlet opening 19 to the deflection region 17, which deflects the flow so that the infusion solution is guided via the step 16 into the downstream portion of the infusion line 3 without detachment. This means that there are no steps/shoulders along the entire flow path where residual air bubbles can adhere. In addition, all deflections are configured as ramps/inclinations or as radii, so that no dead water areas are formed and thus no turbulences and consequently no air bubbles are created in the infusion filter 1 according to the preferred configuration example.

When the flow of the infusion solution stops, the filter membrane 8 remains completely wetted, as described above, and thus forms the air barrier. Residual air in the flow sections upstream of the filter membrane 8 collects in the cavity 7 and can flow out via the vent filter 9 and the elongated hole 27.

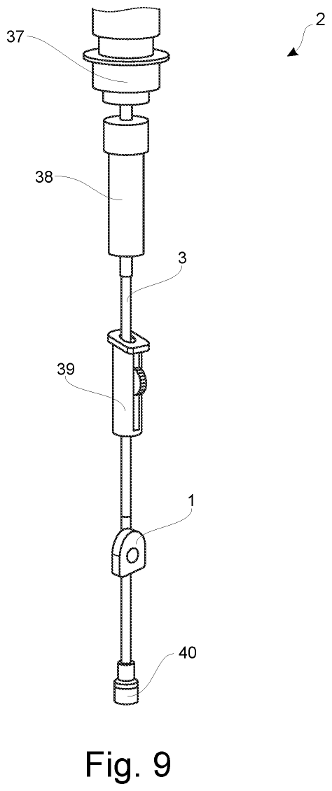

FIG. 9 shows the infusion set 2 with the infusion line 3 and the built-in infusion filter 1 according to the preferred configuration example. A connector 37 for a container for receiving/storing the infusion solution is arranged upstream of the infusion filter 1. Connected to the connector 37 is a drip chamber 38, which is a first device for air separation, and a hose clamp 39 for regulating the flow. Downstream of the infusion filter 1, another (patient) connector 40 is arranged, which can be fluidically connected to an injection site of a patient or another infusion line. In other words, the infusion solution flows from the container via the connector 37 into the infusion line 3, through the drip chamber 38, the hose clamp 39, the infusion filter 1 and the connector 40. The turbulence-free flow through the infusion filter 1 described above makes it possible to arrange the infusion filter 1 in the vicinity of the (patient) connector 40. The arrangement of the infusion filter 1 can be installed at any position in the infusion line 3, depending on the therapy required. It is also possible for the infusion filter 1 to be directly integrated in the connector 40.

In the preferred configuration example described above, the filter membrane 9 is constructed from the set of parallel tubes 11. Alternatively, however, a filter membrane 9 can also be made from a block which has fine gaps running through it in the direction of flow. When the flow stops, the liquid can adhere between these gaps and thus form the air barrier. It is also conceivable that the filter membrane 9 has a plurality of lamellae or is made of an open porous material such as felt or foam.

1. An infusion filter for at least one medical infusion line, the infusion filter comprising:
a housing defining a flow path between a housing inlet and a housing outlet, the housing inlet being couplable to a first portion of the at least one infusion line, and the housing outlet being coupleable to a second portion of the at least one infusion line; and
a filter membrane that is hydrophilic and arranged in the flow path, the filter membrane being aligned parallel to a main flow direction predetermined by the at least one infusion line and placed in the housing in such a way that the filter membrane divides the housing into an upstream housing portion and a downstream housing portion,
the housing comprising, upstream of the filter membrane, a first flow-guiding element that forms a first elbow for a first deflection of a flow along the flow path in a direction transverse to the main flow direction, and a second flow-guiding element forming a baffle surface oriented obliquely to the main flow direction for a second deflection of the flow back into the main flow direction, so as to ensure the flow against the filter membrane exclusively at an upstream membrane side of the filter membrane.

2. The infusion filter according to claim 1, wherein the housing has a third flow-guiding element downstream of the filter membrane that forms a second elbow that deflects the flow flowing substantially vertically through the filter membrane into the main flow direction.

3. The infusion filter according to claim 1, wherein:
the housing inlet has a first receiving region for receiving the first portion of the at least one infusion line,
the housing outlet has a second receiving region for receiving the second portion of the at least one infusion line, and
the first and second receiving regions are adapted to dimensions of the at least one infusion line in such a way that, m-thein a coupled state, inner diameters of the at least one infusion line end flush with surfaces of the receiving regions.

4. The infusion filter according to claim 1, further comprising a vent filter that is air-permeable and arranged in the housing, the vent filter being connected to an outer housing side via an opening formed in the housing.

5. The infusion filter according to claim 1, wherein the housing comprises:
a support structure for receiving or mounting the filter membrane, the support structure having a crosspiece that is closed and follows a geometry of the filter membrane; and
support elements arranged within the crosspiece.

6. The infusion filter according to claim 5, wherein:
the support elements are configured as ribs and knobs,
the ribs are arranged in the main flow direction aligned downstream of the filter membrane directly following filter membrane, and
the knobs are placed downstream of the ribs with respect to the main flow direction.

7. The infusion filter according to claim 1, wherein the filter membrane has a bubble pressure between 0.2 and 0.45 bar and/or an area intended for flow below 2.0 cm².

8. The infusion filter according to claim 1, wherein the filter membrane is constructed from one of:
a plurality of parallel tubes,
a block with gaps,
lamellae, or
an open porous material, a felt or a foam.

9. An infusion set comprising:
the infusion filter according to claim 1; and
an infusion line,
the infusion line comprising a first end portion comprising a first connector for a container with an infusion solution,
the infusion line further comprising a second end portion comprising a second connector for a patient port or a further infusion line.

10. The infusion set according to claim 9, wherein the infusion filter is arranged in a flow direction of the infusion solution from the first end portion to the second end portion directly upstream of the second end portion.

11. The infusion set according to claim 9, wherein the infusion filter is directly integrated in the second connector.

12. The infusion filter according to claim 1, wherein the first deflection of the flow is perpendicular to the main flow direction.

13. The infusion filter according to claim 4, wherein the vent filter is arranged in an upstream housing portion of the housing.

14. The infusion filter according to claim 4, wherein the crosspiece is circular.

15. The infusion filter according to claim 6, wherein the knobs are transversely offset to the ribs with respect to the main flow direction.

16. The infusion filter according to claim 7, wherein the filter membrane has a bubble pressure between 0.2 and 0.45 bar and/or an area intended for flow below 1.7 cm².

17. The infusion filter according to claim 8, wherein the filter membrane is constructed from a membrane.
